(19) United States
(12) Patent Application Publication
(10) Pub. No.: US 2026/0294260 A1
(43) Pub. Date: Oct. 1, 2026

(54) NON-INVASIVE ARTERIAL MONITORING SYSTEM AND METHOD

(71) Applicant: Donald Steven Edelschick, New City, NY (US)

(72) Inventor: Donald Steven Edelschick, New City, NY (US)

(21) Appl. No.: 19/440,812

(22) Filed: Jan. 6, 2026

Related U.S. Application Data
(60) Provisional application No. 63/763,259, filed on Feb. 26, 2025.

(51) Int. Cl. A61B 5/022; A61B 5/00; A61B 5/021; A61B 5/0225; A61B 5/026

(52) U.S. Cl. CPC A61B 5/02233; A61B 5/02125; A61B 5/0225; A61B 5/026; A61B 5/7253; A61B 2560/0223; A61B 2562/0261

(57) ABSTRACT

A non-invasive arterial monitoring system and method are disclosed for obtaining continuous arterial pressure and arterial flow waveforms from a limb. A substantially inelastic strap membrane is configured to encircle the limb and apply controlled circumferential compression using a tensioning actuator. Time-varying arterial pulsations are detected using at least one strain sensor mechanically coupled to the strap membrane and a displacement sensor configured to measure limb expansion. A processor controls a sweep of compression preload values to identify an operating condition that maximizes pulsatile signal fidelity, and computes arterial pressure and arterial flow waveforms from the measured signals. Relationships between the pressure and flow waveforms are analyzed to characterize arterial dynamics and operating regimes. The disclosed system provides high-fidelity, continuous, non-invasive assessment of arterial hemodynamic behavior.

### CROSS-REFERENCE TO RELATED APPLICATIONS

This application claims priority to and the benefit of U.S. Provisional Patent Application No. 63/763,259, filed Feb. 26, 2025, entitled “Non-Invasive Arterial System Monitor,” the entirety of which is incorporated herein by reference.

### FIELD OF THE INVENTION

The present invention relates to non-invasive physiological monitoring and, more particularly, to systems and methods for continuous measurement of arterial pressure and arterial flow waveforms from a limb using controlled circumferential compression and externally applied mechanical sensing.

### SUMMARY OF THE INVENTION

Disclosed is a non-invasive arterial monitoring system and method configured to generate continuous arterial pressure and arterial flow waveforms, including pulsatile (AC) components, from a compressed limb measurement site without arterial cannulation.

In one embodiment, a substantially inelastic strap membrane encircles a limb and overlies at least one artery. A tensioning actuator applies controlled circumferential compression to soft tissue between the strap membrane and the artery. A strain sensor coupled to the strap membrane detects time-varying pulsatile changes in strap strain caused by arterial expansion under compression.

A displacement member mechanically coupled to the strap membrane converts circumferential expansion and contraction of the limb into linear displacement, which is measured by a displacement encoder as a function of time x(t). The measured displacement serves as a surrogate for dynamic changes in limb geometry associated with pulsatile arterial flow.

A processor controls the tensioning actuator to sweep through a plurality of compression preload values and identifies a preload at which a pulsatile arterial signal metric is maximized, thereby optimizing mechanical coupling between the artery and the sensing elements. The identified preload is used to enhance waveform fidelity and stability, and not as a replacement for pulsatile waveform measurement.

Using calibrated relationships, the processor computes a time-varying arterial pressure waveform P(t) from strap strain and a time-varying arterial flow waveform Q(t) from displacement-derived geometric changes. Relationships between P(t) and Q(t) are analyzed to characterize arterial dynamics.

### THEORY OF OPERATION

### Mechanical Coupling and Pulsatile Signal Transduction

During each cardiac cycle, arterial blood flow produces cyclic expansion of the arterial wall. When a limb is encircled by a substantially inelastic strap membrane under controlled circumferential compression, pulsatile arterial expansion is mechanically transmitted through surrounding tissue to the strap membrane.

The strap membrane experiences time-varying tension changes corresponding to pulsatile arterial pressure variations. A strain sensor mechanically coupled to the strap membrane converts these tension changes into an electrical signal representative of pulsatile arterial pressure. The strain sensor responds to pressure transmitted through tissue and does not require direct contact with the arterial wall.

Simultaneously, circumferential expansion and contraction of the limb produce macroscopic changes in limb geometry. These changes are mechanically coupled to a displacement member, such as a piston or plunger, which produces linear displacement x(t) measured by a displacement encoder.

### Compression Preload Sweep and Coupling Optimization

The fidelity of detected pulsatile signals depends on the degree of mechanical coupling between the artery, surrounding tissue, and the strap membrane. Insufficient compression results in poor coupling, while excessive compression attenuates pulsatile motion.

The processor therefore controls the tensioning actuator to sweep through a plurality of compression preload values. At each preload, the processor evaluates a pulsatile arterial signal metric derived from the strain sensor and/or displacement encoder.

The preload at which the pulsatile signal metric is maximized is identified as an operating preload. This operating preload corresponds to a coupling state that enhances waveform fidelity and is maintained during subsequent waveform acquisition.

### Arterial Pressure and Flow Waveform Computation

Time-varying strap strain is converted into an arterial pressure waveform P(t) using a calibrated relationship between strap tension and arterial pressure.

Displacement x(t) is mapped to instantaneous limb radius R(t) and cross-sectional area A(t) using a calibration relationship. A time-varying arterial flow waveform Q(t) is computed as a function of the time derivative of A(t) and/or x(t).

Pulsatile (AC) waveform components are preserved as primary measurement outputs. Time-averaged values are used only as reference descriptors.

### Pressure-Flow Relationship and Energetic Description

The processor analyzes temporal relationships between P(t) and Q(t), including waveform timing, phase relationships, and morphology, which reflect arterial compliance, inertance, wave propagation, and reflection behavior. Accordingly, arterial operating regimes are fundamentally determined by the phase and energy relationships between pressure and flow waveforms, with statistical measures, where used, serving only as normalized representations of underlying physical behavior

Over a defined time window, the processor may compute a mean real hemodynamic power descriptor proportional to the time-average of pressure multiplied by flow. This descriptor provides an additional characterization of arterial energy transfer behavior and does not replace waveform-based analysis.

The system does not assume steady-state or independent-and-identically-distributed conditions. Analysis is performed on finite physiological time windows appropriate for cardiovascular dynamics.

### Dual-Site Measurement (Optional)

In some embodiments, a second sensing unit is positioned along the limb at a known spacing from the first sensing unit. When the spacing corresponds approximately to one quarter of a dominant pulse wavelength, pulse wave velocity and reflection parameters may be computed using measurements from both sensing units.

### DETAILED DESCRIPTION OF EMBODIMENTS

### System Architecture

The non-invasive arterial monitoring system includes a substantially inelastic strap membrane, a tensioning actuator, at least one strain sensor, a displacement member, a displacement encoder, and a processor configured to control actuation and compute arterial waveforms.

The tensioning actuator may comprise a linear pulling solenoid or functionally equivalent mechanism capable of incremental retraction under processor control.

### Displacement Member and Geometry Mapping

The displacement member may comprise a piston or plunger arranged such that circumferential expansion of the limb produces axial motion. The displacement encoder measures x(t), which is mapped to instantaneous limb geometry using a calibration relationship.

### Calibration

Calibration may be performed using one or more of a second strain sensor, a force impulse device configured to apply a known mechanical input to the strap membrane, or reference measurements obtained under controlled conditions.

The processor uses system responses to known inputs to calibrate pressure and flow computation relationships.

### Signal Processing and Analysis

The processor may transform pressure and flow waveforms into the frequency domain to assess impedance-related behavior, phase relationships, and reflection phenomena.

Forward-and reflected-wave components may be separated using known signal processing techniques without requiring invasive measurements.

### Clinical and Research Utility

The system provides continuous, non-invasive arterial pressure and flow waveforms suitable for hemodynamic monitoring, vascular assessment, and cardiovascular research.

1. A non-invasive arterial monitoring system, comprising:
(a) a substantially inelastic strap membrane configured to encircle a limb and overlie at least one artery;
(b) a tensioning actuator mechanically coupled to the strap membrane and configured to incrementally retract a strap segment so as to apply circumferential compression to soft tissue between the strap membrane and the at least one artery;
(c) a first strain sensor mechanically coupled to the strap membrane and positioned to detect time-varying, pulsatile changes in strap strain caused by arterial expansion under the applied compression;
(d) a displacement member mechanically coupled to the strap membrane such that circumferential expansion and contraction of the limb produce time-varying linear displacement of the displacement member;
(e) a displacement encoder configured to measure the displacement as a function of time x(t) as a surrogate for dynamic changes in limb circumference or radius; and
(f) a processor configured to:
(i) control the tensioning actuator to apply a range of compression preload values;
(ii) identify a compression preload at which pulsatile signal amplitude derived from at least one of the first strain sensor and the displacement encoder is maximized;
(iii) compute continuous arterial pressure and arterial flow waveforms from the measured time-varying strain and displacement signals; and
(iv) analyze relationships between the pressure and flow waveforms to characterize arterial dynamics,
whereby the system is configured to generate high-fidelity, continuous, non-invasive arterial pressure and flow measurements, and wherein identification of a compression preload corresponding to a system operating mean is used to enhance measurement fidelity rather than replace pulsatile waveform measurement.

2. A method of non-invasively monitoring arterial hemodynamics, comprising:
(a) encircling a limb with a substantially inelastic strap membrane overlying at least one artery;
(b) applying controlled circumferential compression to the limb using a tensioning actuator mechanically coupled to the strap membrane;
(c) measuring time-varying pulsatile changes in strap strain caused by arterial expansion using a strain sensor coupled to the strap membrane;
(d) measuring time-varying displacement of a displacement member mechanically coupled to the strap membrane using a displacement encoder;
(e) sweeping the applied compression through a plurality of preload values;
(f) identifying a preload value at which pulsatile signal amplitude is maximized;
(g) computing continuous arterial pressure and arterial flow waveforms from the measured time-varying strain and displacement; and
(h) analyzing the computed waveforms to characterize arterial behavior,
wherein identification of the preload value associated with maximal pulsatility is used to improve waveform accuracy and stability, and not as a sole output of the method.

3. The system of claim 1, wherein the tensioning actuator comprises a linear pulling solenoid.

4. The system of claim 3, wherein the processor is configured to maintain the identified compression preload during subsequent waveform acquisition.

5. The system of claim 1, wherein the compression preload is selected to reduce reflection and attenuation of pulsatile arterial signals. Displacement and Geometry

6. The system of claim 1, wherein the displacement member comprises a piston or plunger mechanically coupled to the strap membrane.

7. The system of claim 6, wherein circumferential expansion of the limb produces axial motion of the piston or plunger.

8. The system of claim 1, wherein the processor maps displacement x(t) to instantaneous limb radius and cross-sectional area as functions of time.

9. The system of claim 8, wherein the processor computes arterial flow as a function of a time derivative of limb cross-sectional area.

10. The system of claim 1, wherein arterial pressure is computed from strap strain using a calibrated relationship between strap tension and arterial pressure.

11. In some embodiments, a second strain sensor is coupled to the strap membrane to provide an independent reference measurement of strap strain. The processor compares outputs of the first and second strain sensors to distinguish arterial pressure-induced strain from non-arterial mechanical strain components, including effects of preload tension, strap elasticity, and actuator force transmission. This comparison enables calibration of arterial pressure measurements across varying operating conditions.

12. The system of claim 11, further comprising a force impulse device configured to apply a known mechanical input to the strap membrane.

13. The system of claim 12, wherein the processor calibrates pressure and flow computation using responses to the known mechanical input.

14. The system of claim 1, wherein the processor transforms pressure and flow waveforms into the frequency domain.

15. The system of claim 14, wherein the processor computes impedance or phase relationships between pressure and flow sufficient to distinguish incident and reflected wave components at a measurement location.

16. The system of claim 14, wherein the processor separates forward-propagating and reflected wave components.

17. The system of claim 1, further comprising a second sensing unit positioned at a location spaced from the first sensing unit along the limb.

18. The system of claim 17, wherein the spacing corresponds approximately to one quarter of a dominant pulse wavelength.

19. The system of claim 18, wherein the processor computes pulse wave velocity and reflection parameters using measurements from both sensing units.

20. The system of claim 1, wherein at least one conjugate variable pair associated with the monitored system has an externally reported or previously established mean value.

21. The system of claim 20, wherein the processor correlates measured time-varying values of the conjugate variable pair relative to the established mean value.

22. The system of claim 21, wherein the processor categorizes system behavior into a structural operating regime based on responsiveness of the measured values relative to the established mean.

23. The system of claim 22, wherein the operating regime is classified as at least one of stable, reversionary, expansive, or contractive.

24. The method of claim 2, further comprising maintaining the identified preload during waveform acquisition.

25. The method of claim 2, further comprising transforming pressure and flow waveforms into the frequency domain.

26. The method of claim 2, further comprising separating forward and reflected wave components.

27. The method of claim 2, further comprising correlating measured conjugate variables to an established mean to classify system regime.

28. The system of claim 1, wherein the processor is further configured to compute at least one baseline reference value corresponding to a reference operating state and to compute a deviation of at least one measured waveform feature from the baseline reference value.

29. The system of claim 28, wherein the baseline reference value comprises at least one of:
(i) a time-average of P(t);
(ii) a time-average of Q(t);
(iii) a low-pass-filtered component of P(t) or Q(t);
(iv) a published or externally supplied mean for a conjugate-variable pair; or
(v) a stored reference derived from prior measurements.

30. The system of claim 28, wherein the processor correlates at least one measured conjugate-variable feature to a corresponding established mean to produce a normalized deviation measure used to classify an operating regime.

31. The system of claim 30, wherein the operating regime comprises at least one of stable, reversionary, expansive, or contractive.

32. The system of claim 30, wherein the normalized deviation measure comprises at least one of a z-score, a normalized residual, a ratio to baseline, or a phase-deviation metric relative to a stored reference.

33. The system of claim 30, wherein classifying the operating regime further uses at least one of:
(i) a phase relationship between P(t) and Q(t) or their harmonics;
(ii) an impedance estimate Z(ω)=P(ω)/Q(ω);
(iii) a reflection or mismatch metric computed from measured waveforms; or
(iv) a stability metric computed from temporal evolution of the deviation measure.

34. The method of claim 2, further comprising computing a baseline reference value corresponding to a reference operating state and computing a deviation of at least one measured waveform feature from the baseline reference value.

35. The method of claim 34, further comprising correlating at least one measured conjugate-variable feature to a corresponding established mean to classify an operating regime as stable, reversionary, expansive, or contractive.

36. The system of claim 30, wherein the processor is further configured to compute a mean real power value associated with the conjugate-variable waveforms over a defined time window, and wherein the operating regime is further classified based on a combination of the normalized deviation measure and the mean real power value.

37. The method of claim 35, further comprising computing a mean real power value associated with the conjugate-variable waveforms over a defined time window and using the mean real power value to refine operating-regime classification.
